(19) United States
(12) Patent Application Publication
(10) Pub. No.: US 2023/0039337 A1
(43) Pub. Date: Feb. 9, 2023

(54) INTRACELLULAR MICROLENS AND ITS APPLICATION IN OPTICAL DETECTION AND IMAGING

(71) Applicant: Jinan University, Guangzhou (CN)

(72) Inventors: Yuchao Li, Guangzhou (CN); Baojun Li, Guangzhou (CN); Yao Zhang, Guangzhou (CN); Xixi Chen, Guangzhou (CN)

(73) Assignee: Jinan University, Guangzhou (CN)

(21) Appl. No.: 17/530,210

(22) Filed: Nov. 18, 2021

(30) Foreign Application Priority Data
Aug. 6, 2021 (CN) 2021109030784

(51) Int. Cl. G02B 21/36; G02B 21/16; C12P 7/64

(52) U.S. Cl. CPC G02B 21/361; G02B 21/16; C12P 7/64

(57) ABSTRACT

The invention discloses a microlens and an application thereof, wherein the microlens is a lipid particle. The microlens prepared by the invention is simple in preparation and extraction methods, and does not need extra processing. The lipid particle is capable of serving as an optical element inside a cell to exert an optical function and has a complete biocompatibility; and meanwhile, the lipid particle is naturally generated inside the cell, and has a natural position-close relationship with a microstructure inside the cell, and is capable of collecting and re-positioning an optical signal of the microstructure in a near field, so that an imaging quality of the cell microstructure of an optical microscope is effectively improved.

### CROSS-REFERENCE TO RELATED APPLICATIONS

This application claims the benefit of priority to Chinese Patent Application No. 202110903078.4, filed Aug. 6, 2021, the entire disclosure of which is incorporated herein by reference.

### TECHNICAL FIELD

The invention belongs to the field of optical technologies, and more particularly, relates to a microlens and an application thereof.

### BACKGROUND

Real-time monitoring of changes in internal and external states of a cell by an optical means is of great significance to understand a physiological process and quickly diagnose a cytopathy. A microsphere-assisted technology is widely used in optical experiments due to convenient application and excellent optical performance. This technology not only helps traditional optical microscopes to realize real-time unmarked super-resolution imaging in a visible light band, but also can collect and enhance various optical imaging signals.

However, with the increasing demand and standard of an imaging and detection technology in a biological environment, a biocompatibility of the microsphere-assisted technology in long-term imaging and detection in the biological environment becomes a challenge. In the existing microsphere-assisted technology, commonly used microsphere materials are mostly solid medium microspheres (such as SiO₂, polystyrene, BaTiO₃, TiO₂, etc.). These microsphere materials have a low biocompatibility when applied to the observation of biological samples due to a stable solid characteristic and a low degradability, which may affect a biological activity during long-term use.

### SUMMARY OF THE INVENTION

The invention aims to solve at least one of the above technical problems in the prior art. Therefore, the invention provides an application of a lipid particle in preparing a microlens, which has the advantage of complete biocompatibility, and may be directly applied to a biological environment for microstructure imaging detection inside and outside cells.

The invention further provides a microlens.

The invention further provides an application of the microlens.

The invention further provides a cell imaging method.

According to a first aspect of the invention, an application of a lipid particle in preparing a microlens is provided.

According to a second aspect of the invention, a microlens including a lipid particle extracted from an interior of a cell is provided.

In some embodiments of the invention, the cell is a cell capable of generating a lipid particle with a micrometer size. Preferably, the cell is one of an adipocyte, a cone cell and a stellate cell.

In some embodiments of the invention, the lipid particle extracted from the interior of the cell specifically includes the following steps of: washing the adipocyte or the cone cell with cell buffer, then adding sterile water or sterile water with 0.1% to 1% cell lysis buffer (Triton-X100), and standing for 10 minutes to 30 minutes to obtain the lipid particle.

In some embodiments of the invention, the microlens has a particle size of 1 μm to 40 μm.

According to a third aspect of the invention, an application of the microlens above is provided, wherein the application is an application in preparing a cell micro-optical element.

In some embodiments of the invention, an application of the microlens above in biological imaging is provided.

A cell imaging method includes the following steps of: internalizing the microlens above into a cell to be detected, and performing fluorescence imaging by using the microlens.

In some embodiments of the invention, the microlens stays in the cell for 12 hours to 96 hours.

In some embodiments of the invention, the cell structure includes internal and external microstructures.

According to the embodiments of the invention, the invention at least has the following beneficial effects: according to the invention, the lipid particle is used as the micro-optical element for preparing the microlens, which is an endogenous organelle, and has a higher refractive index than a surrounding cytoplasmic environment. The preparation method is simple, and does not need extra processing. The lipid particle is capable of serving as an optical element inside a cell to exert an optical function and has a complete biocompatibility. Meanwhile, the lipid particle is naturally generated inside the cell, and has a natural position-close relationship with a microstructure (subcellular structure) inside the cell, and is capable of collecting and re-positioning an optical signal of the microstructure in a near field, so that an imaging quality of the cell microstructure of an optical microscope is effectively improved. Moreover, the lipid particle is capable of converging incident excitation light beam to an external environment of the cell, which may realize imaging observation of the external environment of the cell by using the lipid particle inside the cell. The lipid particle may also be combined with an optical tweezers control technology to realize mobile imaging of flexible lipid particle inside the cell. Moreover, the lipid particle is also capable of being extracted by a simple method and applied to other types of cells, thus expanding an application range of the microlens.

### BRIEF DESCRIPTION OF THE FIGURES

The invention is further described hereinafter with reference to the accompanying drawings and the examples, wherein:

FIG. 1 is an optical microscope detection image of lipid particles and mature adipocytes in example 1 of the invention, wherein a is an optical microscopic image of the mature adipocytes; and b is an optical microscopic image of the lipid particles of different sizes extracted from the mature adipocytes;

FIG. 2 is a transmission spectrogram of the lipid particles in example 1 of the invention from visible light to a near-infrared band (400 nm to 1000 nm);

FIG. 3 is an observation result diagram of fluorescence imaging of a fluorescent nano-diamond by a microlens in a test example of the invention, wherein a is a superposition diagram of a bright field of an experimental sample and fluorescence optical microscopy; a1 is a superposition diagram of a bright field of a fluorescent nano-diamond cluster on a glass slide and fluorescence optical microscopy; a2 is a superposition diagram of a bright field of the lipid particles (diameter 9 μm) and fluorescence of the fluorescent nano-diamond cluster after a signal is enhanced; b is a three-dimensional diagram of an intensity of a fluorescence signal in FIG. a; b1 is a three-dimensional diagram of an original fluorescence intensity of the fluorescent nano- diamond (corresponding to FIG. a1); b2 is a three-dimensional diagram of a fluorescence intensity of the fluorescence nano-diamond after enhancement (corresponding to FIG. a2); and c is a diagram of a reduction rate of excitation light power of the microlens;

FIG. 4 is an observation diagram of a subcellular structure inside a cell by the microlens in a test example of the invention, wherein a is a cell microfilament fluorescence image of the mature adipocytes; a1 is a fluorescence image of a target cell microfilament in an optimal focal plane; a2 is a fluorescence image of the target cell microfilament obtained on a virtual image plane of the microlens; b is a normalized curve of intensity distribution of the cell microfilament for target observation obtained in a; c is a fluorescence and optical microscopic image of enhanced imaging of the lysosome in the cells by the lipid particles in the cells; c1 and c2 are respectively fluorescence images focused on a cell surface and on the virtual image plane of the microlens (diameter: 11.3 μm); and c3 is a bright-field optical image focused on the virtual image plane of the microlens (diameter: 11.3 μm).

FIG. 5 is a diagram of an enhancement result of the lipid particles inside the mature adipocytes on a fluorescence signal of leukemia cells in a liquid in a test example of the invention, wherein a is a schematic diagram of a position relationship of an experimental Z-axis, b is a bottom view of experimental observation, a1 is an observation diagram of the leukemia cells in the liquid without entering a range of a focused light beam of the lipid particles; b1 is a fluorescence imaging diagram of mitochondria; a2 is an observation diagram of the leukemia cells gradually entering an imaging range of the lipid particles; b2 is a fluorescence image when the fluorescence signal is collected by the lipid particles; a3 is an observation diagram when the leukemia cells completely enter an axial center of the focused light beam of the lipid particles; and b3 is a fluorescence imaging diagram when the fluorescence signal is collected by the lipid particles.

FIG. 6 is an observation result diagram of the lipid particles with different diameters internalized into phagocytes and tumor cells by biological endocytosis in a test example of the invention, wherein a1 and a2 are diagrams of an observation result of the lipid particles entering the phagocytes; and b1 and b2 are diagrams of an observation result of the lipid particles entering the tumor cells.

### DETAILED DESCRIPTION OF THE PREFERRED EMBODIMENTS

The concept and the generated technical effect of the invention are clearly and completely described hereinafter with reference to the examples to fully understand the objectives, the features and the effects of the invention. Obviously, the described examples are only some but not all of the examples of the invention, and based on the examples of the invention, other examples obtained by those skilled in the art without paying creative work all belong to the scope of protection of the invention.

### Example 1

In this example, a microlens was prepared. The preparation method specifically included the following steps.

1. Culture of Adipocytes

Culture of mature adipocytes: precursor liver adipocytes (precursor subcutaneous adipocytes) were inoculated on a glass culture dish (35 mm) at a density of 1×10³⁻⁴ cell/cm², then added with a precursor adipocyte complete culture medium for culture, and placed in a cell incubator at 37° C. under 5% CO₂ for proliferation for 36 hours. When the precursor adipocytes were 100% in contact with each other, induced differentiation was carried out. The precursor adipocyte complete culture medium was replaced by an adipocyte induced differentiation complete culture medium, and the complete culture medium was replaced once every 2 days. After 6 days, mature adipocytes subjected to induced differentiation could be obtained (an optical microscopic image of the mature adipocytes was shown in FIG. 1a). The mature adipocytes could be cultured in the adipocyte complete culture medium for 5 to 8 days, while spherical lipid particles in the mature adipocytes could exist inside the cells for a long time (5 to 8 days), which was the same as a change in a growth cycle of the cells.

2. Extraction of Lipid Particles Inside Cells

The cell culture medium in the culture dish filled with the mature adipocytes was removed, and the mature adipocytes were washed with 3 mL of cell buffer (Dulbecco's Phosphate Buffered Saline, DPBS) for 3 times. Then, 1 mL of sterile water or sterile water with 0.5% cell lysis buffer (Triton-X100) was added, and stood for 20 minutes, and then floating lipid particles appeared in the solution. A lipid particle solution was obtained by extracting and resuspending a supernatant (an optical microscopic image of the lipid particles with different sizes extracted from the mature adipocytes was shown in FIG. 1B).

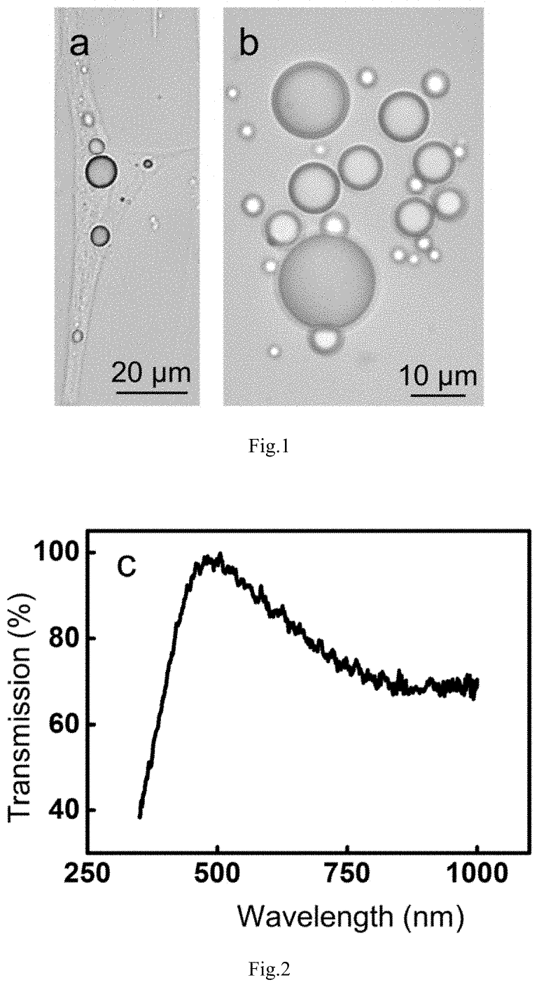

The prepared microlens and the mature adipocytes were detected by an optical microscope. Results are shown in FIG. a and FIG. b. It can be seen from the figures that the microlens is successfully prepared by the solution of the invention. A transmission spectrum of the microlens from visible light to a near-infrared band (400 nm to 1,000 nm) is shown in FIG. 2. It can be seen from the figure that the microlens prepared by the invention has a maximum light transmittance at 500 nm.

### Test Example

1. In-Vitro Optical Imaging Performance of Lipid Particles

An in-vitro optical imaging performance test was carried out on the lipid particles extracted in example 1. The lipid particles were put into a refractive index matching solution of a glycerol solution and water (a refractive index of a matched cytoplasm was 1.36) to test the optical imaging performance. In the experiment, an inverted microscopic imaging optical system matched with an optical tweezers control system was used for observation. A fluorescent sample (a fluorescent nano-diamond and a quantum dot microsphere) which was not easy to be quenched by fluorescence was placed above a sample cavity, and irradiated by excitation light of corresponding wavelengths (red: 590 nm to 650 nm, green: 540 nm to 580 nm, blue: 465 nm to 495 nm) of red, green and blue light-emitting diodes (LED), and an original brightness of the fluorescent sample was compared with that after imaging by using the lipid particles.

In the experiment, the lipid particles with diameters of 1 μm to 40 μm were moved to a position directly below the target fluorescent sample by optical tweezers for imaging observation. The experimental results are shown in FIG. 3. It can be seen from the figure that with the help of the lipid particles, the fluorescence imaging quality of the fluorescent sample observed by the microscope can be effectively improved, and the excitation light power used in the experiment can be reduced.

2. Fluorescence Imaging Observation of Fluorescent Nano-Diamond by Using Extracted Lipid Particles

A fluorescent nano-diamond cluster was observed by using the microlens prepared in example 1, and the fluorescent nano-diamond sample was placed in the sample cavity, and placed above a refractive index matching solution with free lipid particles (50 μL of mixed solution of glycerol and sterile water, with a refractive index of 1.36). Fluorescence excitation of the fluorescent nano-diamond was carried out by using a light source of a light-emitting diode (LED) (wavelength of excitation light: 540 nm to 580 nm), and optical power of the excitation light remained unchanged at ˜2.8 mW.

Experimental results are shown in FIG. 3, wherein a1 is a superposition diagram of a bright field of the fluorescent nano-diamond cluster on a glass slide and fluorescence optical microscopy; b1 is a three-dimensional diagram of an original fluorescence intensity of the fluorescent nano-diamond; a2 is a superposition diagram of a bright field of the lipid particles (diameter 9 μm) and fluorescence of the fluorescent nano-diamond cluster after the lipid particles are imaged; and b2 is a three-dimensional diagram of a fluorescence intensity of the fluorescence nano-diamond after enhancement. By observing with an inverted microscope, only a very weak red fluorescence imaging signal (as shown in a1 and b1) was directly detected from a position of the fluorescent nano-diamond cluster (as shown in a1 and b1). In order to enhance a detection intensity of the fluorescence signal, the lipid particles with a diameter of 9.0 μm were moved below the fluorescent nano-diamond cluster to collect and enhance the fluorescence signal (as shown in a2). An imaging plane of the microscope was adjusted to a virtual imaging plane of the lipid particles. Therefore, an enlarged and enhanced fluorescence imaging image (as shown in b2) was observed.

3. Imaging Detection of Microenvironment Signal Outside Cells by Using Lipid Particles Inside Cells

A focused light beam of an incident light beam formed by the lipid particles inside the cells could be converged outside the cells. In the experiment, a capillary glass microflow tube with a wall thickness of 1 μm to 5 μm and an inner diameter of 10 μm to 45 μm was made by a fused biconical taper method to simulate a capillary in a creature, and placed on a surface of the mature adipocytes closely to simulate a position relationship between a capillary in a tissue and the adipocytes. A center of the capillary was aligned with the lipid particles (diameter>10 μm) in the cells, thus being unified with an observation center of an objective lens. The excitation light irradiated the lipid particles in the cells through an inverted objective lens (with magnification of 60 times, and a numerical aperture of 1), and was converged into the capillary to form strong local excitation light distribution. A fluid of target observation cells with a fluorescence label was introduced into the capillary at a certain speed, the fluid flowed from left to right, and the target observation object moved along with the fluid. A change in an image of the target object during moving was observed by using a microscope. When the target object gradually entered an imaging detection range of the lipid particles, an imaging quality was gradually improved, and a strongest imaging signal was realized when the target object reached the axial center of the converged beam of the lipid particles.

4. Fluorescence Imaging Observation of Subcellular Structure Inside Cells

Fluorescence imaging was carried out on the subcellular structure inside the cells by using the lipid particles inside the cells. The subcellular structure for target observation was fluorescently labeled, so that the subcellular structure could be specifically identified in the experiment. By observing under low excitation light power (1 mW to 2 mW), the target observation object was found under a microscope, and mobile observation was carried out by capturing the lipid particles (diameter: 1 μm to 8 μm) by using an optical potential well generated by the optical tweezers control system (wavelength: 1064 nm, power: 5 mW to 100 mW) or in-situ observation was carried out by directly using the lipid particles (diameter: 1 μm to 40 μm).

5. Observation and Enhancement of Subcellular Structure Inside Cells by Using Lipid Particles Inside Cells

In the test example, imaging observation was carried out on a microstructure (a microfilament and a lysosome) inside the cells by using the lipid particles prepared in example 1 respectively.

Precursor liver adipocytes were inoculated on a glass culture dish (35 mm) at a density of 1×10⁴ cell/cm², then added with a precursor adipocyte complete culture medium for culture, and placed in a cell incubator at 37° C. under 5% CO₂ for proliferation for 36 hours to 48 hours. When the precursor adipocytes were 100% in contact with each other, induced differentiation was carried out. The precursor adipocyte complete culture medium was replaced by an adipocyte induced differentiation complete culture medium, and the complete culture medium was replaced once every 2 to 3 days.

(1) Observation of Cell Microfilament

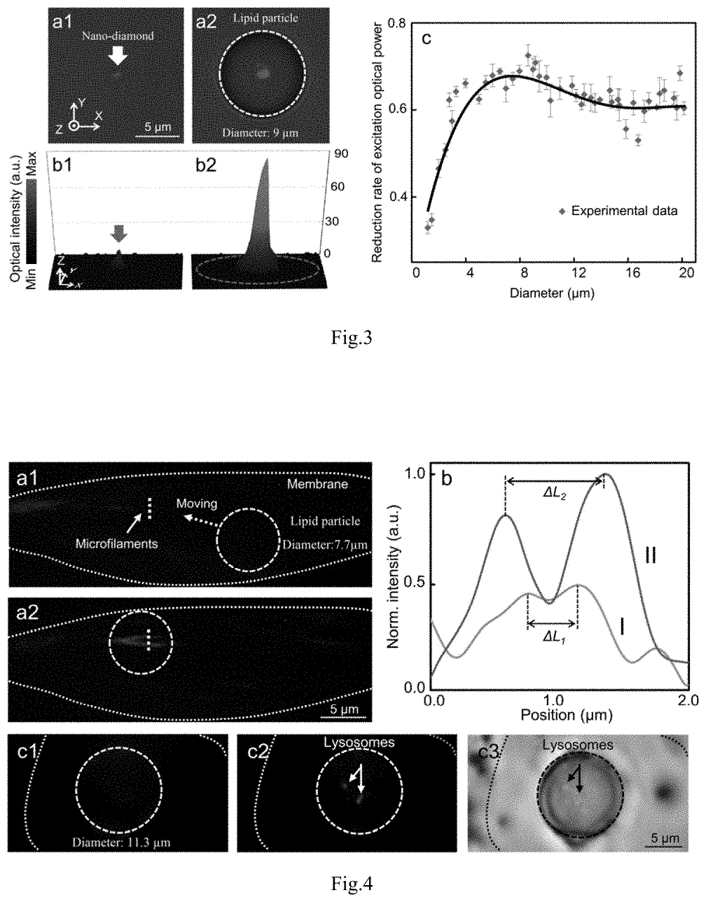

Fluorescence labeling of microfilament structure was carried out on mature adipocyte samples subjected to differentiation for 7 days (dye: SIR-actin, concentration: 0.5 μM, staining time: 30 minutes) and the labeled mature adipocyte samples were rinsed several times (DPBS buffer, 1 mL each time, 2 to 3 times). After staining, the cell samples were placed on an inverted microscope for fluorescence imaging observation (as shown in a1 and a2 in FIG. 4). A wavelength of the excitation light used was 540 nm to 580 nm, and power of the excitation light was kept at 2 mW. As shown in a1 in FIG. 4, an imaging effect of a target observation microfilament inside the cells was blurry under the power of the excitation light so that a structure could not be distinguished. A single optical potential well (wavelength: 1064 nm, power: 20 mW) was added to capture the lipid particles with a diameter of 7.7 μm inside the cells to make the lipid particles move to a position of the target observation microfilament, and a focal plane of the microscope was adjusted to a virtual image plane of the lipid particles. At the moment, a clear structure of the cell microfilament could be seen with the help of the lipid particles.

(2) Observation of Cell Lysosome

Fluorescence labeling of lysosome was carried out on mature adipocyte samples subjected to differentiation for 12 days (stain: Lyso-Tracker, concentration: 0.5 μM, staining time: 30 minutes) and the labeled mature adipocyte samples were rinsed several times (DPBS buffer, 1 mL each time, 2 to 3 times). After staining, the cell samples were placed on an inverted microscope for fluorescence imaging observation. A wavelength of the excitation light used was 540 nm to 580 nm, and power of the excitation light was kept at 1.5 mW.

Experimental results are shown in FIG. 4. It can be seen from the figure that a is a cell microfilament fluorescence image of the mature adipocytes; a1 is a fluorescence image of a target cell microfilament in an optimal focal plane; a2 is a fluorescence image of the target cell microfilament obtained on the virtual image plane of the lipid particles in the cells; b is a normalized curve of intensity distribution of the cell microfilament for target observation obtained in a, a curve I is an original intensity curve of the cell microfilament in a1, and a curve II is an intensity curve of the cell microfilament obtained after enhanced imaging of the lipid particles in the cells in a2; c is a fluorescence and optical microscopic image of enhanced imaging of intracellular lysosomes by the intracellular lipid particles; c1 and c2 are respectively fluorescence images focused on a cell surface and on the virtual image plane of the microlens (diameter: 11.3 μm), and c3 is a bright-field optical image focused on the virtual image plane of the microlens (diameter: 11.3 μm). As shown in c1, when the microscope is directly focused on the cell surface, the lysosome in the cells cannot be directly observed under the microscope because of a weak signal. The lipid particles with a diameter of 11.3 μm inside the cells were selected, and the focal plane of the microscope was adjusted to the virtual image plane of the lipid particles for observation. A clear lysosome image could be observed in both fluorescence (c2) and bright field (c3) imaging modes.

6. Observation and Enhancement of Signal in Fluid Outside Cells by Using Lipid Particles Inside Cells

In the test example, a blood vessel growing around the cells was simulated by using the capillary glass tube, and cancer cells flowing in the capillary glass tube were taken as the target observation object. A fluorescence signal of the cancer cells in the capillary glass tube was detected by using a convergence ability of long excitation light of the lipid particles.

Precursor liver adipocytes were inoculated on a glass culture dish (35 mm) at a density of 1×10⁴ cell/cm², then added with a precursor adipocyte complete culture medium for culture, and placed in a cell incubator at 37° C. under 5% CO₂ for proliferation for 36 hours to 48 hours. When the precursor adipocytes were 100% in contact with each other, induced differentiation was carried out. The precursor adipocyte complete culture medium was replaced by an adipocyte induced differentiation complete culture medium, and the complete culture medium was replaced once every 2 to 3 days. The lipid particles with a large diameter (diameter>10 μm) in the mature adipocytes subjected to differentiation for 12 days could be used as a detection tool by converging a light beam outside the cells.

Leukemia cells were inoculated in a culture flask (25 mm) at a density of 1×10⁴ cell/cm², then added with a complete culture medium (IMDM+10% fetal bovine serum+1% penicillin streptomycin solution) for culture, and placed in a cell incubator at 37° C. under 5% CO₂ for proliferation for 24 hours to 48 hours. 1 mL of leukemia cell suspension was added with Mito-Tracer fluorescent dye (0.1 μL, 5 μM), and stood and stained for 30 minutes. After staining, the leukemia cell suspension was centrifuged (rotating speed: 1000 rpm, time: 5 minutes), then a supernatant was removed, and a complete culture medium (1 mL) was re-added to resuspend the cells.

A microfluidic element was prepared by a fusion draw process to simulate a capillary of a creature. A capillary tube (with an inner diameter of about 0.9 mm, a wall thickness of about 0.1 mm and a length of about 12 cm) was placed in parallel at outer flame above an alcohol lamp, and stood for about 30 seconds. When the glass tube was softened, a softened part was quickly stretched towards two ends at a speed of 6 mm/s by both hands, so as to draw into a glass capillary tube with a wall thickness of 4 μm and an inner diameter of 45 μm. A slender direction of the capillary tube was taken as an experimental area (a length was in a range of 50 um).

A slender part of the prepared glass capillary tube above was placed in the leukemia cell suspension, and the slender part of the capillary tube could be filled with the cell solution under an action of a capillary force. The slender part of the capillary tube was placed on a surface of the mature adipocytes (adherent cells), and a position relationship between a blood vessel in a tissue and the adipocytes was simulated (as shown in 4a). A center of the capillary was aligned with a center of the lipid particles in the adipocytes (diameter=20.0 μm) to match with an observation center of the microscope. The leukemia cell solution (0.1 mL) was sucked with a needle tube (1 mL), and a small amount of suspension was injected into a thick head part of the glass capillary tube, so that a fluid generated in the capillary tube flowed from left to right, thus driving the leukemia cells to move.

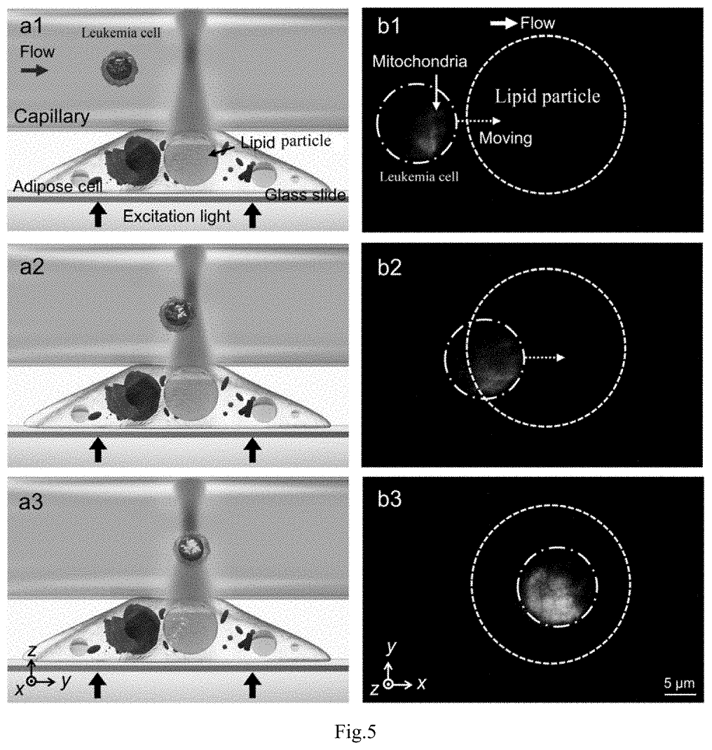

A result of fluorescence imaging of the leukemia cells in the fluid by the lipid particles in the mature adipocytes is shown in FIG. 5. It can be seen from the figure that, by observing under an inverted microscope, when the leukemia cells in the fluid do not enter a range of a focused light beam of the lipid particles (as shown in a1), an observed mitochondrial fluorescence image is weak (as shown in b1). When the leukemia cells gradually enter an imaging range of the lipid particles (as shown in a2), a fluorescence signal is collected by the lipid particles, and an image quality gradually enhanced is observed under a microscope (as shown in b2). When the leukemia cells completely enter an axial center of the focused light beam of the lipid particles (as shown in a3), a fluorescence imaging quality of the leukemia cells is obviously improved (as shown in b3). That is, the leukemia cells gradually flow from an outside of a projection range of the lipid particles (as shown in a1 and b1) to an edge (as shown in a2 and b2) and then to a center (as shown in a3 and b3), during which an intensity of the fluorescence signal is changed.

7. Internalization of Lipid Particles into Other Types of Cells

0.1 mL of lipid particle suspension prepared in Example 1 was added into a complete culture medium solution (2 mL to 5 mL) suitable for other types of cells (cell types capable of occurring cell exocytosis). A supernatant was extracted and the complete medium was re-added for three times, and then a complete culture medium solution with the lipid particles was obtained. When target cells were cultured in a culture dish (35 mm), and a culture density reached 60% to 90%, the original complete culture medium was replaced by the complete culture medium mixed with the lipid particles. The lipid particles and the target cells were co-cultured in a cell incubator at 37° C. under 5% CO₂ for co-culture for 36 hours. Then, a phenomenon and a proportion of internalization of the lipid particles were observed by using an inverted microscope. The internalized lipid particles could stay in other types of cells for 12 hours to 96 hours.

8. Internalization of Extracted Lipid Particles into Phagocytic Nuclear Tumor Cells

The lipid particles prepared in example 1 were introduced into other types of cells as a micro-optical element by biological endocytosis, taking phagocytes and cancer cells as examples.

The phagocytes (Ana-1) were inoculated in a culture flask (25 mm) at a density of 1×10⁴ cell/cm², then added with a complete culture medium (RPMI1640+10% fetal bovine serum+1% penicillin streptomycin solution) for culture, and placed in a cell incubator at 37° C. under 5% CO₂ for proliferation for 24 hours to 36 hours.

The cancer cells were inoculated on a glass culture dish (35 mm) at a density of 1×10⁴ cell/cm², then added with a complete culture medium (DMEM+10% fetal bovine serum+1% penicillin streptomycin solution) for culture, and placed in a cell incubator at 37° C. under 5% CO₂ for proliferation for 36 hours to 48 hours.

Precursor liver adipocytes were inoculated on a glass culture dish (35 mm) at a density of 1×10⁴ cell/cm², then added with a precursor adipocyte complete culture medium for culture, and placed in a cell incubator at 37° C. under 5% CO₂ for proliferation for 36 hours to 48 hours. When the precursor adipocytes were 100% in contact with each other, induced differentiation was carried out. The precursor adipocyte complete culture medium was replaced by an adipocyte induced differentiation complete culture medium, and the complete culture medium was replaced once every 3 days. Mature adipocytes were obtained after differentiation for 12 days. The cell culture medium in the culture dish filled with the mature adipocytes was removed, and the mature adipocytes were washed with 3 mL of cell buffer for 3 times. Then, 1 mL of sterile water was added, and stood for 30 minutes, and then floating lipid particles appeared in the solution.

Two tubes of lipid particle suspension (0.1 mL) were respectively added with a phagocyte complete culture medium and a cancer cell complete culture medium (2 mL). Supernatants were extracted and the complete culture media were re-added for three times to obtain complete culture medium solutions with the lipid particles. The solutions were respectively added into a phagocyte culture flask and a cancer cell culture dish, and placed in a cell incubator at 37° C. under 5% CO₂ for co-culture for 24 hours. After a period of co-culture, by observing under an inverted microscope, the extracted lipid particles successfully entered phagocytes and cancer cells through endocytosis.

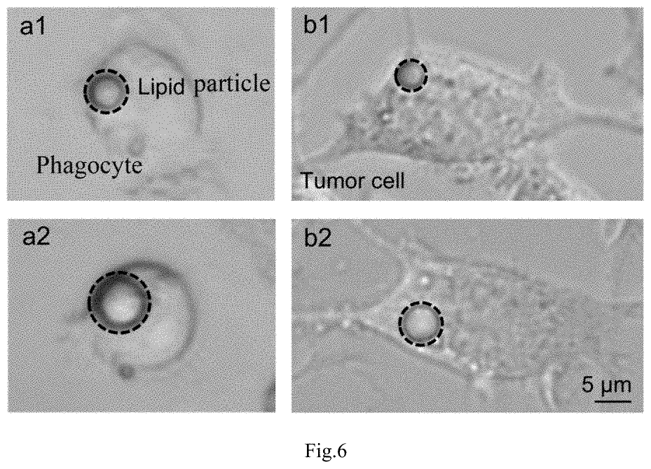

Observation results of the lipid particles co-cultured with the phagocytes and the cancer cells are shown in FIG. 6. It can be seen from the figure that the lipid particles with different diameters (a1 (5 μm); a2 (7.5 μm); b1 (3.3 μm); b2 (5.8 μm)) may all be internalized into the phagocytes (as shown in a1 and a2) and the cancer cells (as shown in b1 and b2) by biological endocytosis, and serve as the microlenses to amplify a fluorescence imaging signal of a microstructure in cells.

The examples of the invention are described in detail with reference to the figures, but the invention is not limited to the above examples, and various changes may also be made within the knowledge scope of those of ordinary skills in the art without departing from the purpose of the invention. In addition, the examples of the disclosure and the features in the examples may be combined with each other without conflict.

1. A method of preparing a microlens, comprising the steps of:
extracting a lipid particle inside a cell;
preparing the lipid particle into a microlens.

2. A microlens comprising a lipid particle extracted from an interior of a cell.

3. The microlens according to claim 2, wherein the cell is a cell capable of generating a lipid particle with a micrometer size.

4. The microlens according to claim 3, wherein the cell is one of an adipocyte, a cone cell and a stellate cell.

5. The microlens according to claim 2, wherein the lipid particle has a particle size of 1 μm to 40 μm.

6. A method of preparing a cell micro-optical element, comprising the steps of:
internalizing the microlens according to claim 2 into a cell to be detected for preparing the cell micro-optical element.

7. A cell imaging method comprising the steps of: internalizing the microlens according to claim 2 into a cell to be detected, and performing cell fluorescence imaging by using the microlens.

8. The method according to claim 7, wherein the microlens stays in the cell to be detected for 12 hours to 96 hours.

9. The method according to claim 7, wherein the cell imaging comprises subcellular structure imaging or cellular microenvironment imaging.
